(19) United States
(12) Patent Application Publication
(10) Pub. No.: US 2018/0136182 A1
(43) Pub. Date: May 17, 2018

(54) ACCURATE MULTI-GAS ANALYZER

(71) Applicant: ORIENTAL SYSTEM TECHNOLOGY INC., Hsinchu (TW)

(72) Inventor: Chien-Hsun WANG, Hsinchu City (TW)

(21) Appl. No.: 15/351,086

(22) Filed: Nov. 14, 2016

(51) Int. Cl. G01N 33/00; G01N 27/02

(52) U.S. Cl. CPC G01N 33/0036; G01N 33/0073; G01N 27/02

(57) ABSTRACT

A multi-gas analysis includes a semiconductor gas sensor driven by a constant resistance driver circuit with a driving current to obtain a sensing output of a gas in a gas sample. A processing unit is operable, based on a reference voltage from the constant resistance driver circuit associated with the driving current, in one of a gas-identification mode, where the gas is identified based on the sensing output obtained in response to fine variation of the operating temperature of the semiconductor gas sensor, and a gas-detection mode, where an analysis result indicative of the concentration of the gas is obtained based on the sensing output obtained in response to an optimal operating temperature of the semiconductor gas sensor.

### FIELD

The disclosure relates to a gas analyzer, and more particularly to a multi-gas analyzer.

### BACKGROUND

Semiconductor gas sensors have been widely applied for detection of gases, such as carbon monoxide (CO), ethanol, methane, propane, butane, hydrogen, etc., due to their characteristics of low cost, long service life and high sensitivity. However, semiconductor gas sensors for one particular gas may have the problem of crosstalk from other gases. For instance, an ethanol semiconductor gas sensor (e.g., available model TG2620) can detect ethanol, but it also responds to hydrogen, isobutene, carbon monoxide and methane with different sensitivities.

In addition, such semiconductor gas sensor is susceptible to ambient temperature effect that adversely affects accuracy of gas concentration measurement.

Therefore, development of a novel multi-gas analyzer, which is inexpensive and capable of identifying and detecting one or more gases contained in a gas sample with high accuracy, is much desirable.

### SUMMARY

Therefore, an object of the disclosure is to provide a multi-gas analyzer that can identify a single gas contained only in a gas sample, that can accurately detect the concentration of the single gas, and that has a compact size and relatively low power consumption.

According to the disclosure, there is provided a multi-gas analyzer for a gas sample that contains a gas. The multi-gas analyzer includes a gas sensor package, a driver unit and a processing unit.

The gas sensor package includes a semiconductor gas sensor that is configured to sense the gas sample in response to receipt of a driving current so as to obtain a sensing output.

The driver unit includes a constant resistance driver circuit that is coupled to the semiconductor gas sensor and that is configured to drive the semiconductor gas sensor with the driving current so as to generate a reference voltage associated with the driving current.

The processing unit is coupled to the semiconductor gas sensor and the constant resistance driver circuit.

The processing unit is operable in one of a gas-identification mode and a gas-detection mode based at least on the reference voltage from the constant resistance driver circuit.

When in the gas-identification mode, the processing unit controls the constant resistance driver circuit to finely vary an operating temperature of the semiconductor gas sensor within a relatively small temperature range, such that the processing unit identifies the gas based on variation of the sensing output, which is obtained from the semiconductor gas sensor in response to the fine variation of the operating temperature of the semiconductor gas sensor.

When in the gas detection mode, the processing unit controls the constant resistance driver circuit to vary the operating temperature of the semiconductor gas sensor to an optimal operating temperature at which the gas is sensed by the semiconductor gas sensor with a relatively high sensitivity, such that the processing unit obtains an analysis result, which includes the concentration of the gas, based at least on the sensing output that is obtained from said semiconductor gas sensor operating at the optimal operating temperature.

### BRIEF DESCRIPTION OF THE DRAWINGS

Other features and advantages of the disclosure will become apparent in the following detailed description of the embodiments with reference to the accompanying drawings, of which:

FIG. 1 is a schematic block diagram illustrating operative relationships among components of a first embodiment of a multi-gas analyzer according to the disclosure;

FIG. 2 is a schematic, partially sectional view exemplarily showing a gas sensor package of the first embodiment;

FIG. 3 is a schematic top view exemplarily showing a first semiconductor gas sensor of the gas sensor package;

FIG. 4 is an equivalent circuit of the first semiconductor gas sensor of the gas sensor package during normal operation;

FIG. 5 is a schematic circuit block diagram exemplarily illustrating the relationship among a heater element of the first semiconductor gas sensor, a first constant resistance driver circuit and a processing unit of the first embodiment;

FIG. 6 is a plot exemplarily illustrating the relationships between sensitivity and operating temperature of an un-doped SnO₂ gas sensor with respect to different annealing temperatures;

FIG. 7 is a plot exemplarily illustrating the relationship between the ambient temperature and variation of sensitivity of the first semiconductor gas sensor;

FIG. 8 is a plot exemplarily illustrating the relationships between sensitivity and operating temperature of the first semiconductor gas sensor when detecting acetone and ethanol simultaneously; and

FIG. 9 is a schematic block diagram illustrating operative relationships among components of a second embodiment of a multi-gas analyzer according to the disclosure.

### DETAILED DESCRIPTION

Before describing the disclosure in greater detail, it should be noted herein that like elements are denoted by the same reference numerals throughout this disclosure. In addition, when two elements are described as being “coupled in series,” “connected in series” or the like, it is merely intended to portray a serial connection between the two elements without necessarily implying that the currents flowing through the two elements are identical to each other and without limiting whether or not an additional element is coupled to a common node between the two elements. Essentially, “a series connection of elements,” “a series coupling of elements” or the like as used throughout this disclosure should be interpreted as being such when looking at those elements alone.

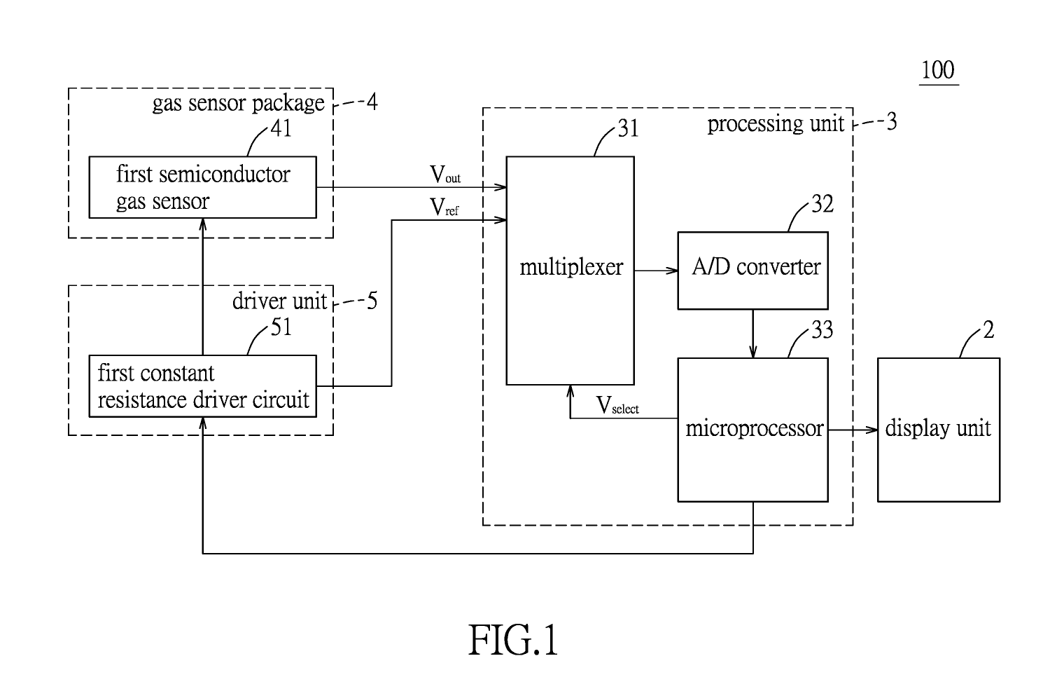

Referring to FIG. 1, the first embodiment of a multi-gas analyzer 100 for a gas sample according to this disclosure is shown to include a gas sensor package 4, a driver unit 5, a processing unit 3 and a display unit 2. It is noted that the gas sample may, for example, only contain a single gas serving as a first gas.

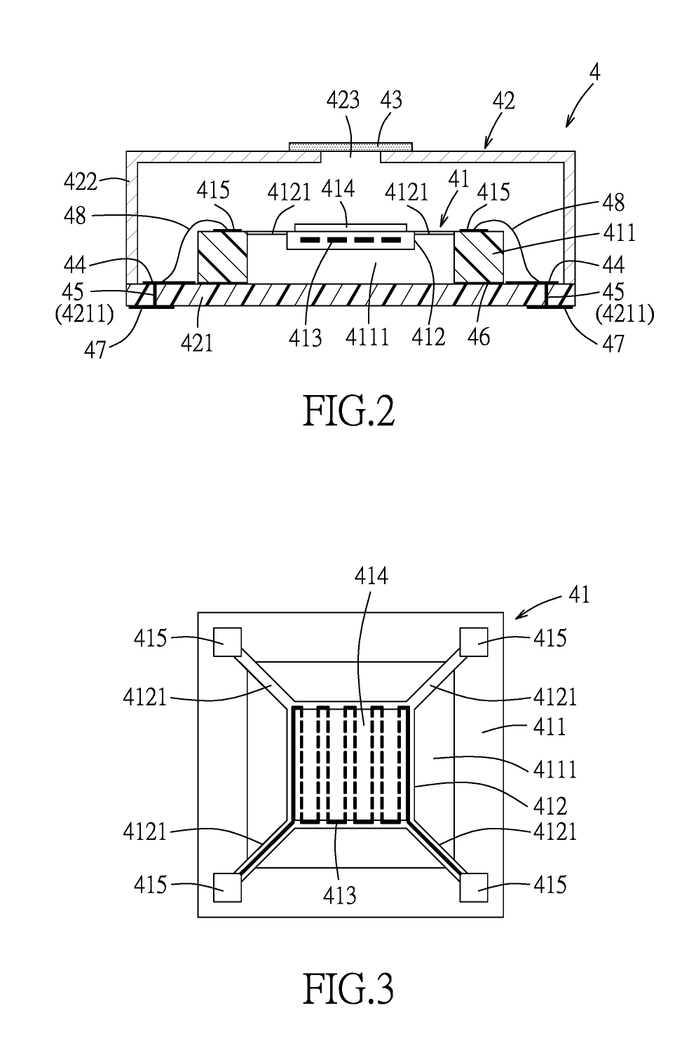

Referring further to FIG. 2, in this embodiment, the gas sensor package 4 may include an enclosure 42, a first semiconductor gas sensor 41 and a dust filter 43.

The enclosure 42 may have a dielectric substrate 421, and a windowed cap 422 connected with the dielectric substrate 421. The dielectric substrate 421 has an inner side surface that is provided with a plurality of conductive pads 44 thereon, an outer side surface that is opposite to the inner side surface and that is provided with a plurality of conductive pads 47 thereon. The conductive pads 44 generally correspond respectively to the conductive pads 47 in position. The dielectric substrate 421 is formed with a plurality of through holes 4211 that extend from the inner side surface to the outer side surface and are filled with conductive material 45. The through holes 4211 are arranged in a manner that each conductive pad 44 is connected electrically to a respective conductive pad 47 through the conductive material 45 filled in a corresponding through hole 4211. The windowed cap 422 is formed with a window opening 423.

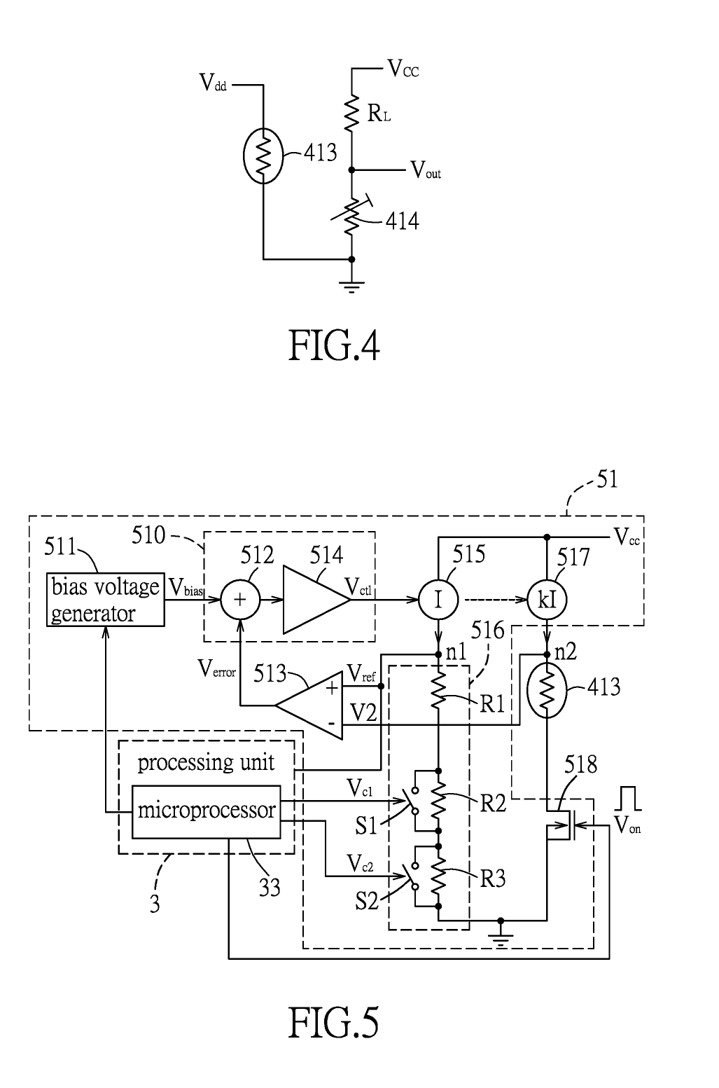

The first semiconductor gas sensor 41 is configured to sense the gas sample in response to receipt of a first driving current so as to obtain a first sensing output. Referring further to FIG. 3, the first semiconductor gas sensor 41 may be in the form of a gas sensor die, and exemplarily includes a silicon base 411, a membrane 412, a heating element 413 and a sensor element 414. The silicon base 411 has a top surface provided with a plurality of conductive pads 415 thereon, a bottom surface attached to the dielectric substrate 421 of the enclosure 42 with an adhesive 46, and a central cavity 4111 extending from the top surface to the bottom surface. The membrane 412 is suspended in the central cavity 4111, and has four radially extending columns 4121. The conductive pads 415 are connected respectively to the columns 4121, and are connected electrically to corresponding conductive pads 44 through bonding wires 48. The heater element 413 is disposed on the membrane 412, receives the first driving current, and is driven by the first driving current to heat the sensor element 414 to an optimal operating temperature. The sensor element 414 is disposed above the heater element 413 with dielectric isolation. As a result, each of the heater element 413 and the sensor element 414 is connected electrically to corresponding conductive pads 47 through corresponding conductive pads 415 and corresponding bonding wires 48. In this embodiment, the heater element 413 is, for example, a microelectromechanical systems (MEMS)-based heater element. FIG. 4 exemplarily illustrates an equivalent circuit of the first semiconductor gas sensor 41 during normal operation. From FIG. 4, a heater voltage (V_{dd}) is applied to the heater element 413, and a sensor voltage (V_{cc}) is applied to a series connection of a load resistor (R_{L}) and the sensor element 414. In this case, the heater voltage (V_{dd}) is associated with the first driving current, and a voltage across the sensor element 414 serves as the first sensing output (Vₒᵤₜ).

The dust filter 43 is attached externally to the windowed cap 422 to cover sealingly the window opening 423, and is configured to filter out dust from the gas sample.

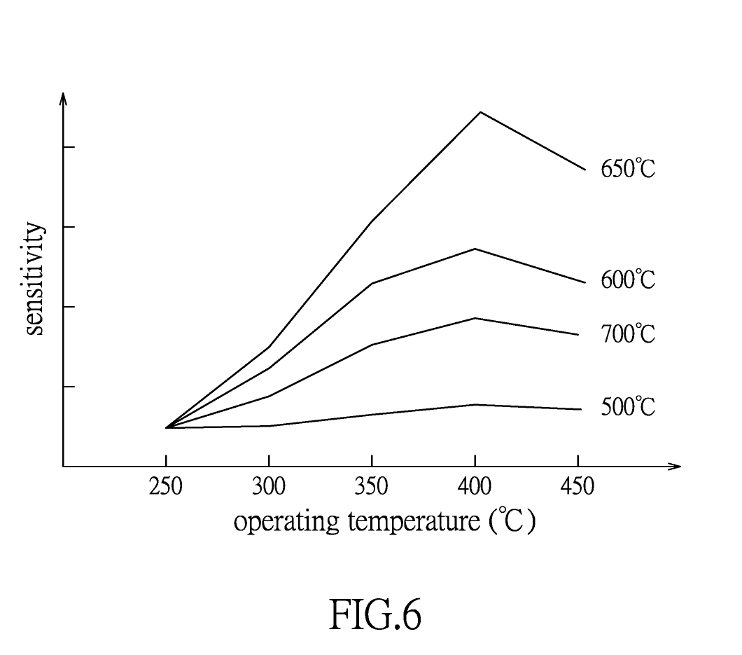

It is noted that sensitivity of the first semiconductor gas sensor 41 depends on operating temperature and annealing temperature of the same. FIG. 6 exemplarily illustrates the relationships between sensitivity and operating temperature of the first semiconductor gas sensor 41 being an un-doped SnO₂ gas sensor at annealing temperatures of 500° C., 600° C., 650° C. and 700° C. From FIG. 6, the first semiconductor gas sensor 41, when implemented as an un-doped SnO₂ gas sensor, may have optimal sensitivity when it was fabricated at the annealing temperature of 650° C. and when it operates at the operating temperature of 400° C.

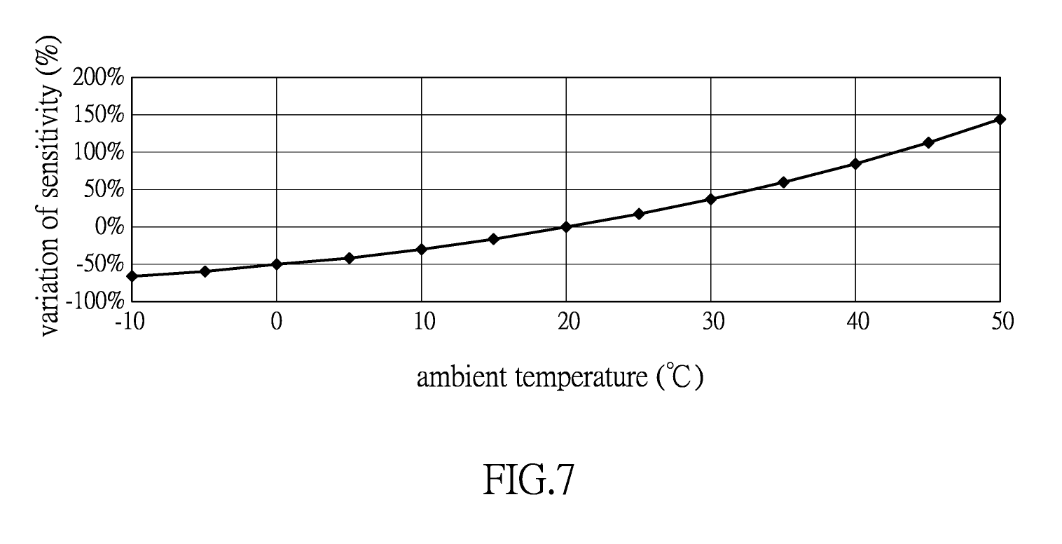

On the other hand, under application of a fixed voltage, the sensitivity of the first semiconductor gas sensor 41 may also vary due to change in ambient temperature, for example, as reported in TGS8100 MEMS sensor datasheet. For the same gas to be sensed, in the exemplary case that a gas concentration of 30 ppm is sensed by the first semiconductor gas sensor 41 under the ambient temperature of 20° C., FIG. 7 exemplarily illustrates the relationship between the ambient temperature and variation of the sensitivity of the first semiconductor gas sensor 41. Therefore, in order to stabilize the sensitivity of the first semiconductor gas sensor 41, it is important for the first semiconductor gas sensor 41 to be unaffected by the ambient temperature effect.

Referring again to FIG. 1, in order to overcome the above ambient temperature effect for the first semiconductor gas sensor 41, the driver unit 5 exemplarily includes a first constant resistance driver circuit 51 that is coupled to the first semiconductor gas sensor 41, for example, through external wiring and the corresponding conductive pads 47 (FIG. 2), for providing the first driving current and that is configured to drive the first semiconductor gas sensor 41 with the first driving current so as to generate a first reference voltage (V_{ref}) associated with the first driving current.

Referring further to FIG. 5, in this embodiment, the first constant resistance driver circuit 51 includes a bias voltage generator 511, a summing amplifier 510, a voltage comparator 513, a voltage controlled current source 515, a mirrored current source 517, a changeable reference sensing resistor unit 516 and a switch 518.

The voltage controlled current source 515 receives a supply voltage (V_{cc}) and is configured to generate a reference current (I) based on a control voltage (V_{ctl}).

The mirrored current source 517 receives the supply voltage (V_{cc}) and is coupled to the heater element 413 for supplying thereto the driving current that is k times the reference current (I), i.e., kI, where k is an amplification factor.

The changeable reference sensing resistor unit 516 is coupled to the voltage controlled current source 515 for receiving the reference current (I) therefrom. The changeable reference sensing resistor unit 516 is controlled to have a desired reference resistance so as to generate the first reference voltage (V_{ref}), which is a voltage across the changeable reference sensing resistor unit 516. In this embodiment, the changeable reference sensing resistor unit 516 may include a series connection of first, second and third reference resistors (R1, R2, R3) that is coupled between the voltage controlled current source 515 and ground, and first and second reference switches (S1, S2) that are coupled respectively to the second and third resistors (R2, R3) in parallel. For example, the second reference resistor (R2) may have a resistance greater than that of the third resistor (R3). Each of the first and second reference switches (S1, S2) may be, for example, an NMOS transistor (not shown), and has a control end for receiving a respective control signal (V_{c1}, V_{c2}) such that each of the first and second reference switches (S1, S2) is operable to be conducting or non-conducting in response to the respective control signal (V_{c1}, V_{c2}). It is noted that, in some embodiments, the changeable reference sensing resistor unit 516 may include four or more different resistors coupled in series to each other, and three or more reference switches as required.

The switch 518 is coupled between the heater element 413 of the first semiconductor gas sensor 4 and ground. In use, the switch 518 is controlled to be conducting so as to allow supply of the driving current (kI) from the mirrored current source 517 to the heater element 413. In this embodiment, the switch 518 is, for example, an NMOS transistor that has a control end for receiving a control voltage (Vₒₙ) such that the NMOS transistor is operable to be conducting or non-conducting in response to the control voltage (Vₒₙ).

The voltage comparator 513 has a non-inverting input end coupled to a first common node (n1) between the voltage controlled current source 515 and the changeable reference sensing resistor unit 516 for receiving the first reference voltage (V_{ref}), an inverting input end coupled to a second common node (n2) between the mirrored current source 517 and the heater element 413 for receiving a voltage (V2) across the heater element 413, and an output end. The voltage comparator 513 compares the first reference voltage (V_{ref}) and the voltage (V2) to generate a comparison output (Vₑᵣᵣₒᵣ), and outputs the comparison output (Vₑᵣᵣₒᵣ) at the output end.

The bias voltage generator 511 is configured to generate a bias voltage (V_{bias}).

The summing amplifier 510 is coupled to the voltage controlled current source 515, the output end of the voltage comparator 513 and the bias voltage generator 511. The summing amplifier 510 is configured to generate the control voltage (V_{ctl}) based on the comparison output (Vₑᵣᵣₒᵣ) from the voltage comparator 513 and on the bias voltage (V_{bias}) from the bias voltage generator 511. In this embodiment, the summing amplifier 510 includes an adder 512 coupled to the bias voltage generator 511 and the output end of the voltage comparator 513 for receiving the bias voltage (V_{bias}) and the comparison output (Vₑᵣᵣₒᵣ) therefrom, and an operational amplifier 514 coupled between the adder 512 and the voltage controlled current source 515 for generating the control voltage (V_{ctl}) based on the bias voltage (V_{bias}) and the comparison output (Vₑᵣᵣₒᵣ). In this case, the control voltage (V_{ctl}) can be expressed as the following equation:

V_{ctl}=m1×V_{bias}+(1−m1)×Vₑᵣᵣₒᵣ,

where m1 is a bias parameter.

In such a configuration of the first constant resistance driver circuit 51, the voltage comparator 513 can maintain the first reference voltage (V_{ref}) to be equal to the voltage (V2). That is to say, I×R_{ref}=kI×Rₕₑₐₜₑᵣ, where R_{ref} represents the reference resistance of the changeable reference sensing resistor unit 516 and Rₕₑₐₜₑᵣ represents the resistance of the heater element 413, and hence Rₕₑₐₜₑᵣ=R_{ref}/k. It is noted that, since the changeable reference sensing resistor unit 516 has the known reference resistance (R_{ref}), the resistance (Rₕₑₐₜₑᵣ) of the heater element 413 can be maintained at a fixed known value of R_{ref}/k. Therefore, the heater element 413 can normally operate at a fixed operating temperature regardless of changes in the ambient temperature to thereby stabilize the operating temperature of the sensor element 414.

The processing unit 3 is coupled to the first semiconductor gas sensor 41 and the first constant resistance driver circuit 51. The processing unit 3 is operable in one of a gas-identification mode and a gas-detection mode.

When in the gas-identification mode, the processing unit 3 controls the first constant resistance driver circuit 51 to finely vary an operating temperature of the heater element 413 of the first semiconductor gas sensor 41 within a relatively small temperature range, such that the processing unit 3 identifies the only gas contained in the gas sample based on variation of the first sensing output (Vₒᵤₜ), which is obtained from the first semiconductor gas sensor 41 in response to the fine variation of the operating temperature of the first semiconductor gas sensor 41.

When in the gas detection mode, the processing unit 3 controls the first constant resistance driver circuit 51 to vary the operating temperature of the heater element 413 of the first semiconductor gas sensor 41 to a corresponding optimal operating temperature at which the identified gas is sensed by the first semiconductor gas sensor 41 with a relatively high sensitivity, such that the processing unit 3 obtains an analysis result, which includes the concentration of the identified gas, based on the first sensing output (Vₒᵤₜ) that is obtained from the first semiconductor gas sensor 41 operating at the corresponding optimal operating temperature.

In this embodiment, for example, the processing unit 3 includes a multiplexer 31, an analog-to-digital (A/D) converter 32 and a microprocessor 33 (FIG. 1).

The multiplexer 31 is connected electrically to the first semiconductor gas sensor 41 and the first constant resistance driver circuit 51 for receiving the first sensing output (Vₒᵤₜ) and the first reference voltage (V_{ref}) respectively therefrom. The multiplexer 31 is operable to output, in response to a selection control signal (V_{select}), the first sensing output (Vₒᵤₜ) and the first reference voltage (V_{ref}) one by one.

The A/D converter 32 is connected electrically to the multiplexer 31 for receiving the first sending output (Vₒᵤₜ) and the first reference voltage (V_{ref}) therefrom. The A/D converter 32 is configured to convert the first sensing output (Vₒᵤₜ) and the first reference voltage (V_{ref}) respectively into a digital first sensing signal and a digital first reference voltage in a high resolution manner. In this embodiment, the A/D converter 32 is, for example, a sigma-delta-type A/D converter. Thus, the digital voltage output may have more than 14 bits.

The microprocessor 33 is connected electrically to the multiplexer 31, the A/D converter 32 and the first constant resistance driver circuit 51, and receives from the A/D converter 32 the digital first sensing signal and the digital first reference voltage (FIG. 1). The microprocessor 33 is configured to provide the selection control signal to the multiplexer 31.

In this embodiment, referring again to FIG. 5, the microprocessor 33 is further configured to control operations of the switch 518 and the first and second reference switches (S1, S2) of the changeable reference resistor unit 516. The microprocessor 33 is connected electrically to the control ends of the switch 518, and the first and second reference switches (S1, S2) for outputting the control voltage (Vₒₙ), and the control signals (V_{c1}, V_{c2}) respectively thereto. It is noted that the operations of the first and second reference switches (S1, S2) are associated with of the gas identification mode and the gas detection mode of the processing unit 3.

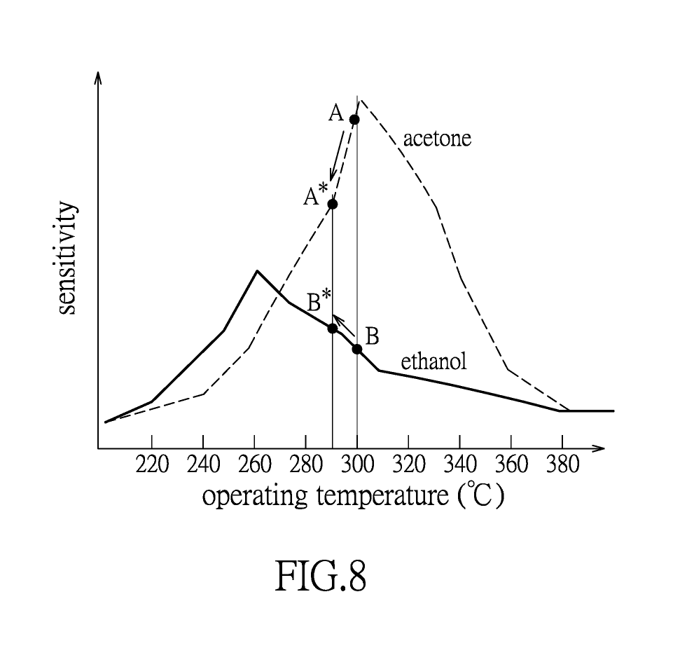

When the processing unit 3 operates in the gas-identification mode, the microprocessor 33 controls operations of the first and second reference switches (S1, S2) of the changeable reference sensing resistor unit 516 of the first constant resistance driver circuit 51 using the control signals (V_{c1}, V_{c2}) provided thereby (see FIG. 5). In this case, the microprocessor 33 thus controls the fine variation of the operating temperature of the first semiconductor gas sensor 41, and calculates, based on the digital first sensing signal from the A/D converter 32, for example, a delta slope of the sensitivity of the first semiconductor gas sensor 41 serving as the variation of the first sensing output (Vₒᵤₜ) for the identified gas contained in the gas sample in response to the fine variation in the operating temperature of the heater element 413 of the first semiconductor gas sensor 41. Since different gases, such as acetone and ethanol, may have individual variations of the sensitivity within a specific temperature range of, for example, from 300° C. to 290° C., as shown in FIG. 8, the only gas contained in the gas sample (i.e., the first gas) can be identified based on the variation calculated by the processing unit 3.

After the first gas, which is the sole constituent of the gas sample, has been identified, the processing unit 3 switches from the gas identification mode to the gas detection mode. When the processing unit 3 operates in the gas detection mode, for the identified first gas, the microprocessor 33 controls the first constant resistance driver circuit 51 to vary the operating temperature of the first semiconductor gas sensor 41 to a corresponding optimal operating temperature at which the identified gas is sensed by the first semiconductor gas sensor 41 with a relatively high sensitivity, such that the microprocessor 33 obtains the analysis result, which includes the concentration of the identified first gas, based on the first sensing output (Vₒᵤₜ) that is obtained from the first semiconductor gas sensor 41 operating at the corresponding optimal operating temperature.

The display unit 2 is connected electrically to the microprocessor 33 (FIG. 1) for displaying the analysis result thereon.

As an example of the first gas contained in the gas sample being acetone or ethanol, in use, initially, the processing unit 3 operates in the gas identification mode for identifying the first gas. In this stage, the first and second reference switches (S1, S2) are first controlled by the microprocessor 33 to be non-conducting in response respectively to the control signals (V_{c}, V_{c2}). Thus, since the reference resistance (R_{ref}) of the changeable reference sensing resistor unit 516 is maximum, the resistance (Rₕₑₐₜₑᵣ) (=R_{ref}/k) of the heater element 413 is also maximum such that the first semiconductor gas sensor 41 operates at a relatively high operating temperature of, for example, 300° C. In this case, the microprocessor 33 may obtain, based on the digital first sensing signal from the A/D converter 32 that corresponds to the current first sensing output (Vₒᵤₜ) obtained from the first semiconductor gas sensor 41 under the operating temperature of 300° C., the sensitivity of the first semiconductor gas sensor 41 with respect to the first gas (i.e., acetone or ethanol, for example), as indicated by one of points (A, B) in FIG. 8.

Thereafter, the first and second reference switches (S1, S2) are controlled by the microprocessor 33 to respectively be non-conducting and conducting in response to the control signals (V_{c1}, V_{c2}), respectively. Thus, since the reference resistance (R_{ref}) of the changeable reference resistor unit 516 decreases, the resistance (Rₕₑₐₜₑᵣ) also decreases. As a result, the operating temperature of the heater element 413 is lowered to about 290° C., for example. It is noted that the second reference switch (S2) switching from non-conducting to conducting or from conducting to non-conducting can be regarded as a fine tune for the operating temperature of the heater element 413. In this case, the microprocessor 33 may also obtain, based on the digital first sensing signal from the A/D converter 32 that corresponds to the current first sensing output (Vₒᵤₜ) obtained from the first semiconductor gas sensor 41 under the operating temperature of 290° C., the sensitivity of the first semiconductor gas sensor 41 with respect to the first gas (i.e., acetone or ethanol), for example, as indicated by one of points (A*, B*) in FIG. 8. Therefore, if the first gas is acetone, the microprocessor 33 may calculate variation of the sensitivity of the first semiconductor gas sensor 41 for acetone in response to the fine variation of the operating temperature of the first semiconductor gas sensor 41, i.e., a delta slope of a dash line segment A-A* of FIG. 8. Alternatively, if the first gas is ethanol, similarly, the microprocessor 33 may calculate variation of the sensitivity of the first semiconductor gas sensor 41 for ethanol in response to the fine variation of the operating temperature of the first semiconductor gas sensor 41, i.e., a delta slope of a solid line segment B-B* in FIG. 8. Since the delta slope of the dash line segment A-A* and the delta slope of the solid line segment B-B* match respectively two sensitivity-to-operating temperature characteristic curves of the first semiconductor gas sensor 41 with respect to acetone and ethanol, as indicated respectively by a dash curve and a solid curve in FIG. 8, the first gas can thus be identified based on the variation of the sensitivity of the first semiconductor gas sensor 41 in response to the fine variation of the operating temperature (i.e., from 300° C. to 290° C.) of the first semiconductor gas sensor 41.

Then, the processing unit 3 operates in the gas detection mode for obtaining the analysis result that includes the concentration of the first gas. In this stage, for detection of the first gas, for example, if the first gas is acetone, the microprocessor 33 may obtain the concentration of acetone based on the digital first sensing signal from the A/D converter 32 that corresponds to the first sensing output (Vₒᵤₜ) obtained from the first semiconductor gas sensor 41 under the operating temperature of 300° C. That is to say, the optimal operating temperature for detection of acetone is, for example, 300′C. Alternatively, if the first gas is ethanol, the first and second reference switches (S1, S2) are controlled by the microprocessor 33 to respectively be conducting and non-conducting in response to the control signals (V_{c1}, V_{c2}), respectively. Since the resistance of the second reference resistor (R2) is designed to be greater than the resistance of the third reference resistor (R3), the reference resistance (R_{ref}) of the changeable reference sensing resistor unit 516 further decreases such that the operating temperature of the heater element 413 may be further lowered to, for example, about 260° C., which is the optimal operating temperature for detection of ethanol (see FIG. 8). It is noted that the first reference switch (S1) switching from non-conducting to conducting or from conducting to non-conducting can be regarded as a coarse tune for the operating temperature of the heater element 413. Thus, the microprocessor 33 may obtain the concentration of ethanol based on the digital first sensing signal from the A/D converter 32 that corresponds to the first sensing output (Vₒᵤₜ) obtained from the first semiconductor gas sensor 41 under the operating temperature of 260° C. The concentration of the first gas obtained by the microprocessor 33 constitutes the analysis result.

From the above example, the changeable reference sensing resistor unit 516 of the first constant resistance driver circuit 51 may be appropriately designed as required for analyzing different desired gases.

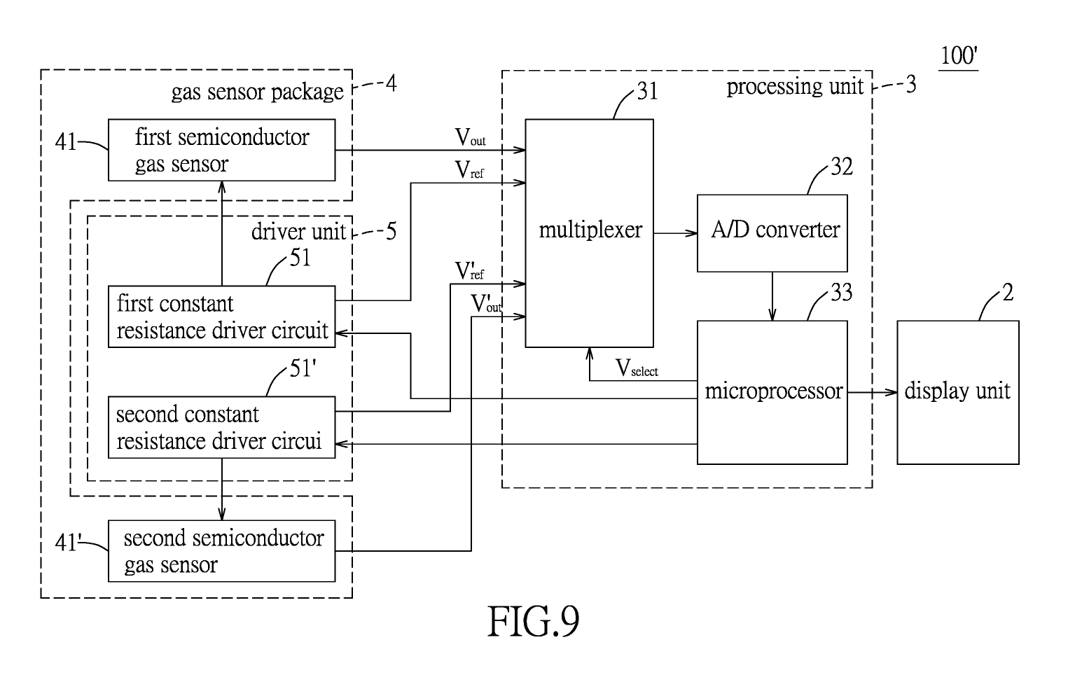

FIG. 9 illustrates the second embodiment of a multi-gas analyzer 100′ for at most two gas samples containing respectively the first gas and a second gas different from the first gas according to this disclosure, which is a modification of the first embodiment.

In this embodiment, the gas sensor package 4 further includes a second semiconductor gas sensor 41′ housed in the enclosure 42 (FIG. 2) for sensing the gas sample that contains the second gas in response to receipt of a second driving current to obtain a second sensing output (V′ₒᵤₜ). The second semiconductor gas sensor 41′ may be similar to the first semiconductor gas sensor 41 in structural configuration.

In addition, the driver unit 5 further includes a second constant resistance driver circuit 51′ coupled to the second semiconductor gas sensor 41′ for driving the second semiconductor gas sensor 41′ with the second driving current to generate a second reference voltage (V′_{ref}) associated with the second driving current. In this embodiment, the second constant resistance driver circuit 51′ may be similar to the first constant resistance driver circuit 51 in structural configuration.

In view of the changes made in the gas sensor package 4 and the driver unit 5, the multiplexer 31 of the processing unit 3 is connected electrically further to the second semiconductor gas sensor 41′ and the second constant resistance driver circuit 51′ for receiving the second sensing output (V′ₒᵤₜ) and the second reference voltage (V′_{ref}) respectively therefrom, such that the multiplexer 31 outputs to the A/D converter 32, in response to the selection control signal from the microprocessor 33, the first and second sensing outputs (Vₒᵤₜ, V′ₒᵤₜ) and the first and second reference voltages (V_{ref}, V′_{ref}) one by one.

Thus, the A/D converter 32 of the processing unit 3 further receives the second sensing output (V′ₒᵤₜ) and the second reference voltage (V′_{ref}) from the multiplexer 31, and further converts the second sensing output (V′ₒᵤₜ) and the second reference voltage (V′_{ref}) respectively into a digital second sensing signal and a digital second reference voltage in the high resolution manner.

In this embodiment, the microprocessor 33 of the processing unit 3 is connected electrically further to the second constant resistance driver circuit 51′. When the processing unit 3 operates in the gas-identification mode, the microprocessor 33 is configured further to control the second constant resistance driver circuit 51′ to finely vary an operating temperature of the second semiconductor gas sensor 41′ within another relatively small temperature range in a manner similar to that for the first constant resistance driver circuit 51, and calculate, based on the digital second sensing signal from the A/D converter 32, variation of the sensitivity of the second semiconductor gas sensor 41′ for the second gas in response to the fine variation in the operating temperature of the second semiconductor gas sensor 41′ so as to identify the second gas based on the variation calculated thereby. When the processing unit 3 operates in the gas-detection mode, for the identified second gas, the microprocessor 33 is configured further to control the second constant resistance driver circuit 51′ to vary the operating temperature of the second semiconductor gas sensor 41′ to a corresponding optimal operating temperature at which the identified second gas is sensed by the second semiconductor gas sensor 4′ with a relatively high sensitivity, and analyze the digital second sensing signal from the A/D converter 32 in response to the corresponding optimal operating temperature of the second semiconductor gas sensor 41′ to obtain the concentration of the identified second gas to be included in the analysis result.

It is noted that, since the first semiconductor gas sensor 41 and the first constant resistance driver circuit 51 are used for identification and detection of the first gas, and since the second semiconductor gas sensor 41′ and the second constant resistance driver circuit 51′ are used for identification and detection of the second gas, the multi-gas analyzer 100′ can be configured to identify and detect the gas samples, which contain respectively the first and second gases, at the same time, thereby improving gas-identification and gas-detection efficiency.

To sum up, through control of the changeable reference sensing resistor unit 516 of each of the first and second constant resistance driver circuits 51, 51′ by the microprocessor 33, the multi-gas analyzer 100, 100′ can easily identify the gas(es) contained (respectively) in the gas sample(s) when the processing unit 3 operates in the gas-identification mode, and accurately detect each identified gas when the processing unit 3 operates in the gas-detection mode. In particular, since the first and second constant resistance driver circuits 51, 51′ implement constant-resistance driving respectively for the first and second semiconductor gas sensors 41, 41′ under quick analog feedback control by the microprocessor 33, each of the first and second semiconductor gas sensors 41, 41′ can operate at a desired fixed operating temperature during use. Moreover, since the first and second semiconductor gas sensors 41, 41′ fabricated using MEMS technology have a relatively small footprint and relatively low power consumption, the gas sensor package 4 may be in the form of a surface mount device (SMD) package. Therefore, the multi-gas analyzer 100, 100′ of this disclosure can be fabricated to have a compact size and relatively low power consumption.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A multi-gas analyzer for a gas sample that contains a first gas, said multi-gas analyzer comprising:
a gas sensor package including a first semiconductor gas sensor that is configured to sense the gas sample in response to receipt of a first driving current so as to obtain a first sensing output;
a driver unit including a first constant resistance driver circuit that is coupled to said first semiconductor gas sensor and that is configured to drive said first semiconductor gas sensor with the first driving current so as to generate a first reference voltage associated with the first driving current; and
a processing unit coupled to said first semiconductor gas sensor and said first constant resistance driver circuit, said processing unit being operable in one of a gas-identification mode and a gas-detection mode;
wherein said processing unit controls, in the gas-identification mode, said first constant resistance driver circuit to finely vary an operating temperature of said first semiconductor gas sensor within a relatively small temperature range, such that said processing unit identifies the first gas based on the first sensing output that is obtained from said first semiconductor gas sensor in response to the fine variation of the operating temperature of said first semiconductor gas sensor, and controls, in the gas-detection mode, said first constant resistance driver circuit to vary the operating temperature of said first semiconductor gas sensor to an optimal operating temperature at which the first gas is sensed by said first semiconductor gas sensor with a relatively high sensitivity, such that said processing unit obtains an analysis result, which includes the concentration of the first gas, based at least on the first sensing output that is obtained from said first semiconductor gas sensor operating at the optimal operating temperature.

2. The multi-gas analyzer as claimed in claim 1, wherein said processing unit includes:
a multiplexer connected electrically to said first semiconductor gas sensor and said first constant resistance driver circuit for receiving the first sensing output and the first reference voltage respectively therefrom, said multiplexer being operable to output, in response to a selection control signal, the first sensing output and the first reference voltage one by one;
an analog-to-digital (A/D) converter connected electrically to said multiplexer for receiving the first sensing output and the first reference voltage therefrom, said A/D converter being configured to convert the first sensing output and the first reference voltage respectively into a digital first sensing voltage and a digital first reference voltage in a high resolution manner; and
a microprocessor connected electrically to said multiplexer, said A/D converter and said first constant resistance driver circuit, said microprocessor being configured to
provide the selection control signal to said multiplexer,
control the fine variation of the operating temperature of said first semiconductor gas sensor and calculate, based on the digital first sensing voltage from said A/D converter, variation of the sensitivity of said first semiconductor gas sensor for the first gas in response to the fine variation in the operating temperature of said first semiconductor gas sensor so as to identify the first gas based on the variation of the sensitivity of said first semiconductor gas sensor calculated thereby when said processing unit operates in the gas-identification mode, and
analyze the digital first sensing voltage from said A/D converter in response to the optimal operating temperature of said first semiconductor gas sensor to obtain the analysis result when said processing unit operates in the gas-detection mode.

3. The multi-gas analyzer as claimed in claim 2, wherein said A/D converter is a sigma-delta-type A/D converter, and each of the digital first sensing voltage and the digital first reference voltage has more than 14 bits.

4. The multi-gas analyzer as claimed in claim 2, further comprising a display unit connected electrically to said microprocessor of said processing unit for displaying the analysis result thereon.

5. The multi-gas analyzer as claimed in claim 2, wherein:
said first semiconductor gas sensor includes a heater element coupled to said first constant resistance driver circuit for receiving the first driving current therefrom, and a sensor element coupled to said multiplexer of said processing unit, said heater element being driven by the first driving current to heat said sensor element so that said sensor element generates the first sensing output; and
said first constant resistance driver circuit includes
a voltage controlled current source configured to generate a reference current based on a control voltage,
a mirrored current source coupled to said heater element of said first semiconductor gas sensor and configured to supply the first driving current that is k times the reference current,
a changeable reference sensing resistor unit coupled to said voltage controlled current source for receiving the reference current therefrom, and controlled by said microprocessor of said processing unit to have a desired reference resistance so as to generate the first reference voltage, which is a voltage across said changeable reference sensing resistor unit,
a voltage comparator configured to compare the first reference voltage and a voltage across said heater element of said first semiconductor gas sensor to generate a comparison output,
a bias voltage generator for generating a bias voltage, and
a summing amplifier coupled to said voltage controlled current source, said voltage comparator and said bias voltage generator, said summing amplifier being configured to generate the control voltage based on the comparison output from said voltage comparator and the bias voltage from said bias voltage generator.

6. The multi-gas analyzer as claimed in claim 5, wherein said first constant resistance driver circuit of said driver unit further includes a switch coupled between said heater element of said first semiconductor gas sensor of said gas sensor package and ground, said switch being controlled by said microprocessor of said processing unit to allow supply of the first driving current from said mirrored current source to said heater element of said first semiconductor gas sensor.

7. The multi-gas analyzer as claimed in claim 6, wherein said switch is an NMOS transistor.

8. The multi-gas analyzer as claimed in claim 5, wherein:
said changeable reference sensing resistor unit of said first constant resistance driver circuit of said driver unit includes
a series connection of at least first, second and third reference resistors that is coupled between said voltage controlled current source and ground, said second reference resistor having a resistance greater than that of said third resistor, and
a first reference switch and a second reference switch that are coupled respectively to said second and third resistors in parallel; and
said microprocessor of said processing unit further controls operation of each of said first and second reference switches to decide the desired resistance of said changeable reference resistor unit.

9. The multi-gas analyzer as claimed in claim 8, wherein each of said first and second reference switches is an NMOS transistor.

10. The multi-gas analyzer as claimed in claim 5, wherein said heater element of said first semiconductor gas sensor is a microelectromechanical systems (MEMS)-based heater element.

11. The multi-gas analyzer as claimed in claim 2, wherein said gas sensor package further includes:
an enclosure that houses said first semiconductor gas sensor therein and that is formed with a window opening; and
a dust filter attached to said enclosure to cover sealingly said window opening, and configured to filter out dust from the gas sample.

12. The multi-gas analyzer as claimed in claim 11, for another gas sample containing a second gas different from the first gas together with the gas sample, wherein:
said gas sensor package further includes a second semiconductor gas sensor housed in said enclosure for sensing the gas sample that contains the second gas in response to receipt of a second driving current to obtain a second sensing output;
said driver unit further includes a second constant resistance driver circuit coupled to said second semiconductor gas sensor for driving said second semiconductor gas sensor with the second driving current to generate a second reference voltage associated with the second driving current;
said multiplexer of said processing unit is connected electrically further to said second semiconductor gas sensor and said second constant resistance driver circuit for receiving the second sensing output and the second reference voltage respectively therefrom, such that said multiplexer outputs to said A/D converter, in response to the selection control signal from said microprocessor, the first and second sensing outputs and the first and second reference voltages one by one;
said A/D converter of said processing unit further receives the second sensing output and the second reference voltage from said multiplexer, and further converts the second sensing output and the second reference voltage respectively into a digital second sensing signal and a digital second reference voltage in the high resolution manner; and
said microprocessor of said processing unit is connected electrically further to said second constant resistance driver circuit of said driver unit, and is configured further to
when said processing unit operates in the gas-identification mode, control said second constant resistance driver circuit to finely vary an operating temperature of said second semiconductor gas sensor within another relatively small temperature range, and calculate, based on the digital second sensing voltage from said A/D converter, variation of the sensitivity of said second semiconductor gas sensor for the second gas in response to the fine variation in the operating temperature of said second semiconductor gas sensor so as to identify the second gas based on the variation of the sensitivity of said second semiconductor gas sensor calculated thereby, and
when said processing unit operates in the gas-detection mode, control said second constant resistance driver circuit to vary the operating temperature of said second semiconductor gas sensor to an optimal operating temperature at which the second gas is sensed by said second semiconductor gas sensor with a relatively high sensitivity, and analyze the digital second sensing signal from said A/D converter in response to the optimal operating temperature of said second semiconductor gas sensor to obtain the analysis result including the concentration of the second gas.

13. The multi-gas analyzer as claimed in claim 12, wherein said second semiconductor gas sensor is similar to said first semiconductor gas sensor in structural configuration, and said second constant resistance driver circuit is similar to said first constant resistance driver circuit in circuit configuration.
